(19) United States
(12) Patent Application Publication
(10) Pub. No.: US 2022/0175451 A1
(43) Pub. Date: Jun. 9, 2022

(54) LUMINESCENCE PROBE FOR IN VIVO TEMPERATURE MEASUREMENT AND CONTROL

(71) Applicant: GYRUS ACMI, INC. D/B/A OLYMPUS SURGICAL TECHNOLOGIES AMERICA, Westborough, MA (US)

(72) Inventor: Sergey A. Bukesov, Acton, MA (US)

(21) Appl. No.: 17/541,721

(22) Filed: Dec. 3, 2021

Related U.S. Application Data
(60) Provisional application No. 63/120,793, filed on Dec. 3, 2020.

(51) Int. Cl. A61B 18/26

(52) U.S. Cl. CPC A61B 18/26; A61B 2018/00982

(57) ABSTRACT

Various examples disclosed relate to temperature monitoring of medical probes. The present disclosure includes a medical device including a medical probe and one or more luminescent marks. The medical probe can include a distal portion configured for at least partial insertion into a patient. The one or more luminescent marks can be located on the distal portion of the probe and have a luminescent characteristic correlative to temperature, when illuminated. The luminescent characteristic can provide an indication of the temperature at an internal site of the patient.

### CROSS-REFERENCE TO RELATED APPLICATION

This application claims the benefit of priority to U.S. Provisional Patent Application Ser. No. 63/120,793, filed Dec. 3, 2020, the contents of which are incorporated herein by reference in their entirety.

### BACKGROUND

The present disclosure generally relates to both surgical probes for treatment of various anatomical areas, and endoscopes for imaging and/or providing passage of therapeutic devices toward various anatomical portions, including gastrointestinal tract (e.g., esophagus, stomach, duodenum, pancreaticobiliary duct, intestines, colon, and the like), renal area (e.g., kidney(s), ureter, bladder, urethra) and other internal organs (e.g., reproductive systems, sinus cavities, submucosal regions, respiratory tract), and the like.

Monitoring of such surgical probes and endoscope during treatment can be desired. Specifically, the internal anatomical site(s) being treated may be subject to temperature changes during treatment. Monitoring the temperature of those internal sites can be beneficial.

### SUMMARY OF THE DISCLOSURE

Discussed herein are methods and systems for monitoring and detecting temperature at an endoscopic or other internal procedure site within a patient's body, such as by monitoring temperature-dependent luminescence therein. The luminescence can be analyzed to determine temperature, for example, by detecting or monitoring parameters of luminescence itself, or determining a chance of monitored luminescence. Luminescence detection can be compared with stored information about luminescence related to temperature, such as to determine temperature or temperature changes. The stored information about the relationship between luminescence and temperature can take various forms, such as a lookup table, a fitted function, or other data.

In a first aspect, a device can include a medical probe having one or more luminescent marks thereon to monitor temperature of the probe and surrounding environment.

In a second aspect, a system can include a medical probe having one or more luminescent marks thereon, the one or more luminescent marks configured to monitor temperature of the probe and surrounding environment, a laser fiber to provide a laser beam for medical treatment, a light source for providing light to the one or more luminescent marks, a camera for detecting a luminescence response signal from the one or more luminescent marks and providing a resulting electrical signal indicative of the detected luminescence, and signal processing circuitry for analyzing the resulting electrical signal indicative of the detected luminescence and generating a resulting indication of temperature at the internal site being monitored.

In a third aspect, a system can include a medical probe having one or more luminescent marks thereon, the one or more luminescent marks configured to be monitored to indicate a temperature of the probe and surrounding environment, a laser fiber to provide a laser beam for medical treatment, a light source for providing light to the one or more luminescent marks, a camera for detecting a luminescence response signal from the one or more luminescent marks and providing a resulting electrical signal indicative of the detected luminescence, and signal processing circuitry for analyzing the resulting electrical signal indicative of the detected luminescence and generating a resulting indication of temperature at the internal site being monitored, wherein the one or more luminescent marks can be located on the laser fiber.

In a fourth aspect, a system can include a medical probe having one or more luminescent marks therein, the one or more luminescent marks configured to monitor temperature of the probe and surrounding environment, a laser beam for medical treatment, a light source for providing light to the one or more luminescent marks, a camera for detecting feedback from the one or more luminescent marks, and a feedback analyzer, wherein the one or more luminescent marks is located on an end of the probe.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIGS. 1A-1B illustrate an example of a surgical probe.

FIG. 2 illustrates schematic view of a surgical probe temperature measurement system including a luminescent mark.

FIG. 3 illustrates a schematic view of a surgical probe temperature measurement system including a luminescent mark.

FIG. 4 illustrates a schematic view of a surgical probe temperature measurement system including a luminescent mark.

### DETAILED DESCRIPTION

The present disclosure describes, among other things, the use of one or more luminescent marks on a medical probe such as to allow for monitoring and control of temperature at or near a working region of such a medical probe or endoscope.

Luminescence is a spontaneous emission of light by a substance in response to excitation energy, as opposed to heating. Examples of luminescence can include fluorescence and phosphorescence. Both organic and inorganic substances can exhibit luminesce. For example, phosphors convert energy into electromagnetic radiation in the visible light range and produce luminescence.

Luminescence can be sensitive to temperature, such as phosphor thermometry, which can leverage optical methods for temperature measurement. In some cases, such luminescence can be used to control and measure temperature indirectly, such as by measuring temperature-dependent light emitting parameters of luminescent materials, including light intensity, emission spectra range and shape, rise and decay times, and other parameters. The emission decay time or afterglow parameters can be used for temperature measurement.

Luminescence can be useful to track the temperature at the internal procedure site within the patient's body, such as in an example laser lithotripsy procedure. In such an example, monitoring temperature can help protect against necrosis or other issues that temperature extremes or large temperature changes may present. The methods and systems discussed herein can help provide a “non-contact” method to check ambient temperature at or near an internal target region at which the medical procedure is occurring.

For example, temperature-dependent luminescence can be monitored at an internal procedure site, such as when a distal working portion of the medical instrument approaches or meets the target tissue. Luminescence can be used to indirectly measure temperature, such as by measuring one or more temperature-dependent light emitting parameters of luminescence. Examples of temperature-dependent light emitting parameters of luminescence can include luminescence light intensity, luminescence emission spectra range or shape, luminescence rise and decay times, combinations thereof, and others. There are many organic and inorganic luminescent materials that exhibit a change in one or more luminescence parameters in a characterizable relationship to a temperature change, including a temperature change in a range that can occur during a laser lithotripsy or similar medical procedure.

In some cases, the use of luminescence for temperature monitoring and control can be done without requiring additional wiring along the probe for performing a luminescence-based temperature measurement. For example, one or more small (e.g., about 100 micrometers) luminescent marks can be incorporated into a distal portion of a probe. In some cases, luminescent marks can be placed in a variety of places along a probe, such as on a laser fiber, on the distal end of a scope portion of the probe, or in multiple places.

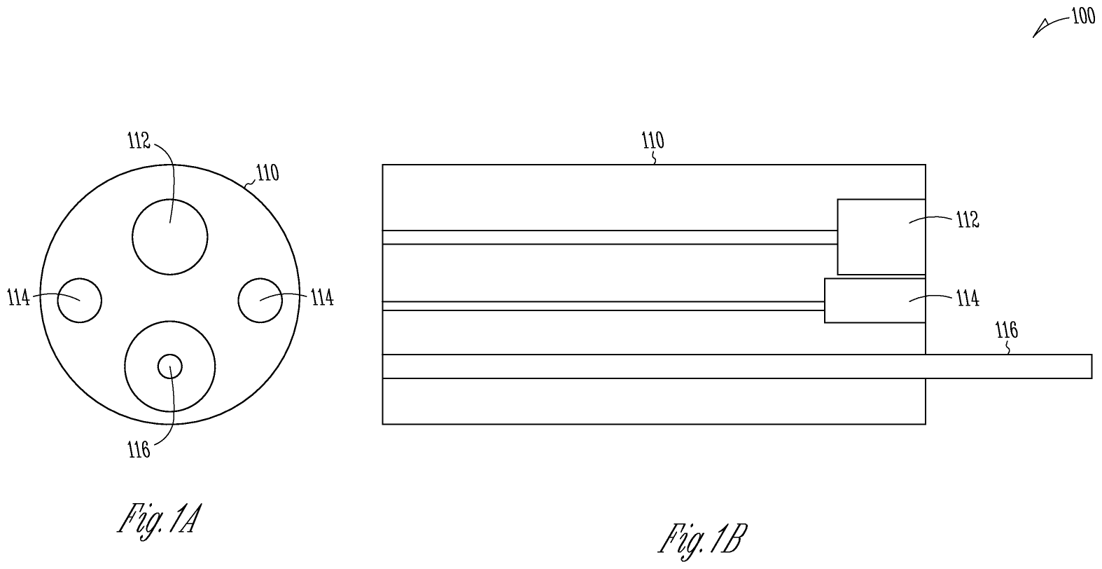

FIGS. 1A and 1B depict an example system 100 with a probe 110 that can use luminescent marks for temperature monitoring in the system 100. In an example, the system 100 can include a probe 110, a camera 112, a light source 114, and a laser fiber 116. In the system 100, the camera 112, the light source 114, and the laser fiber 116 can extend along the length of the probe 110. FIG. 1A depicts a cross-sectional view of the system 100, while FIG. 1B depicts a side schematic view of the system 100.

The probe 110 can extend between a proximal end and a distal end. The probe 110 can be sized, shaped, or arranged for partial insertion into a patient, such as during surgery, for imaging and/or to provide passage of one or more therapeutic devices or sampling devices. The probe 110 can, for example, be involved in a variety of clinical procedures, including, for example, illuminating, imaging, detecting and diagnosing one or more disease states, providing fluid delivery (e.g., saline or other preparations via a fluid channel) toward an anatomical region, providing passage (e.g., via a working channel) of one or more therapeutic devices for sampling or treating an anatomical region, and providing suction passageways for collecting fluids (e.g., saline or other preparations) and the like.

The probe 110 can be couple to an imaging and control system, such as the camera 112, light source 114, and laser fiber 116. The camera 112, light source 114, and laser fiber 116 can be used to providing imaging data to the user. The camera 112 can be a surgical camera suitable for use with the probe 110. The camera 112 can be used, for example, for monitoring an anatomical sight during a procedure. In some cases, the camera 112 may be constructed as being similar to the Olympus Corporation's Endocapsule® endoscopy system. In system 100, the camera 112 can, in some cases, be capable of detecting luminesce or light of particular wavelengths, either visible or non-visible, such as discussed in more detail below.

The light source 114 can be a source capable of producing electromagnetic radiation in a desired wavelength range. The light source 114 can be used to illuminate the anatomical region using light of desired spectrum (such as one or more of broadband white light, narrow-band imaging using preferred electromagnetic wavelengths, or the like). The light source 114 can be configured for production of visible light (e.g., about 380 nm to about 760 nm) within the probe 110 and the surrounding anatomical area. In some cases, the light source 114 can be configured to produce light at a specific wavelength to induce luminescence in one or more luminescent marks as discussed in more detail below. Additional optical components (lens assemblies and/or prism) for illumination and/or collection of image signals can be included in the light source 114. One or more optical fibers (e.g., fiber bundle) may optically couple the illumination optics to the light source 114.

In some cases, the camera 112 and the light source 114 can be partially or fully mounted within or on the probe 110, as desired. The camera 112 or the light source 114 can, in some cases, be connected to a control system or a computer. The camera 112 or the light source 114 can interface with the probe 110 by wired or wireless electrical connections. The light source 114, in conjunction with a controller or computer, can accordingly illuminate an anatomical region, and the camera 112 can collect signals representing the anatomical region. The controller can process the collected signals representing the anatomical region, and can display images representing the anatomical region on a display. Such a controllers can connect (e.g., via an endoscope connector) to the probe 110 for signal transmission (e.g., light output from light source, video signals from imaging system in the distal end, and the like).

The laser fiber 116 can include a single or a bundle of laser fibers extending along the probe 110, such as in a lumen therein, or along a side of the probe 110. The laser fiber 116 can be, for example, any appropriate surgical laser, such as a carbon dioxide laser (e.g., with a wavelength of about 9,000 nm to about 11,000 nm), a diode laser (e.g., with a wavelength in the range of about 800 nm to about 1,100 nm), or an erbium laser (e.g., with a wavelength in the range of about 2,500 nm to about 3,000 nm). In some cases, the laser fiber 116 can be used for surgical treatment.

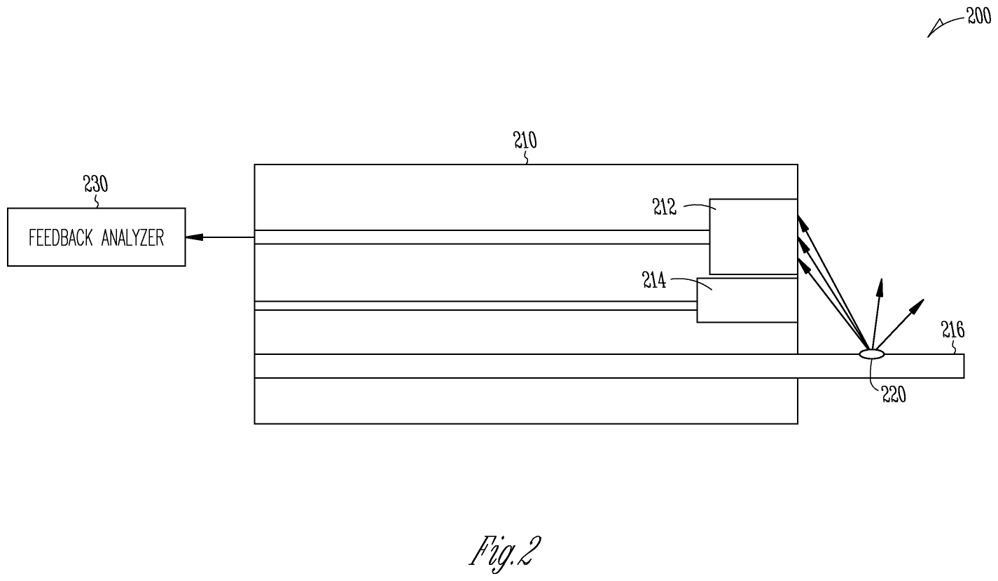

The system 100 can, for example, be used for surgical treatment of tissue, such as with the laser. During treatment, the surgeon (or other user) can desire to monitor temperature at the anatomical site being treated. Monitoring temperature can help protect against necrosis or other issues that temperature extremes or large temperature changes may present. Shown and discussed with reference to FIGS. 2-4, a luminescent mark can be used with such an endoscope or probe to aid in monitoring of temperature during use.

In such a system, an optical response signal from the luminescent probe can be collected through the surgical fiber or a probe camera and a resulting electrical signal can be analyzed by signal processing circuitry that can include a calibrated response signal temperature analyzer. FIGS. 2-4 depict examples of a temperature measurement system in the form of small luminescent mark placed into the surgical probe or laser fiber. Light emission of a luminescent mark presented in FIGS. 2-4 can be activated by different ways depending on chosen luminescent types.

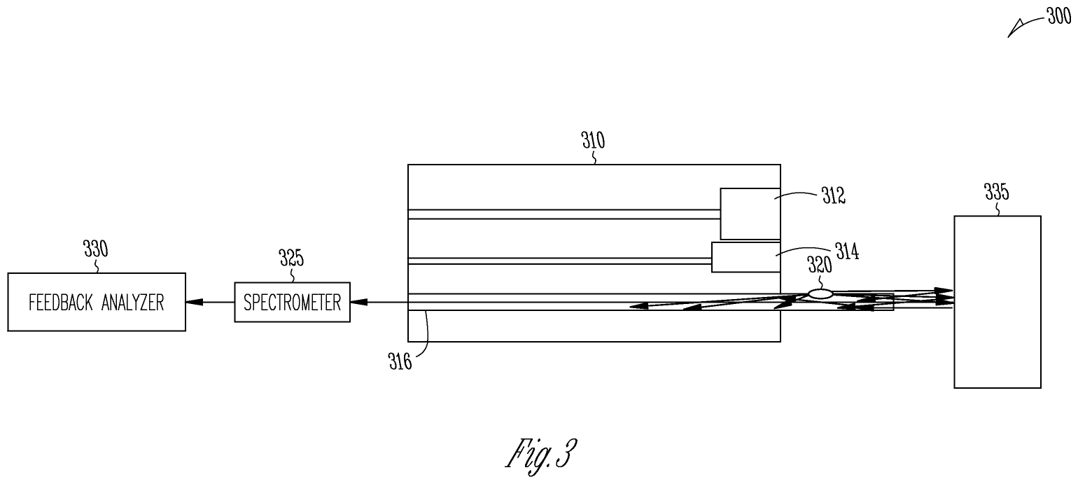

In FIGS. 2 and 3, an example luminescent mark is shown on a laser fiber in a medical probe system. FIG. 2 depicts an example surgical system 200. In an example, the system 200 can include n probe 210, a camera 212, a light source 214, a laser fiber 216, a luminescent mark 220, and a feedback analyzer 230. The components of system 200 can be similar to, and connected in a similar fashion, to the corresponding components discuss with reference to FIGS. 1A-1B above, except where otherwise noted.

In the system 200, the camera 212, light source 214, and the laser fiber 216 can extend at least partially within the probe 210. The luminescent mark 220 can be located on the laser fiber 216. When in used, the laser fiber 216 can be energized by a light source 214, and the luminescent mark 220 can be energized by the light source 214. The camera 212 can capture produced luminescence, which can be correlated to a temperature of the system 200.

Similar to the system 100 above, the light source 214 can be, for example, a visible light source to light up the tissue, and to allow for camera images, in addition to activating the luminescent mark 220. The camera 212 can capture the luminescence and visible images. The produced luminescence can be processed by signal processing circuitry such as to compare to a previously determined luminescence-to-temperature relationship to determine the temperature of the tissue. In some examples, light from the luminescent mark can be captured from the laser fiber itself.

The luminescent mark 220 can be one or more luminescent material pieces on or around the probe 210. The luminescent mark 220 can be a single mark or multiple marks. In system 200, the shown luminescent mark 220 can be located on the laser fiber 216. The luminescent marks 220 can be located in any part of the surgical system 200 for example, endoscope surface, fiber core, cladding, buffer or jacket. In some cases, in the system 200, the luminescent mark 220 can be placed on the laser fiber 216, or alternatively can be placed in the fiber core or cladding, on the end of the scope, near the input of a suction lumen, elsewhere within the probe 210, or combinations thereof. The placement of luminescent marks can be selected to allow better temperature distribution determination throughout the system 200. For example, an average temperature can be monitored throughout the system 200 where luminescent marks are placed on the laser fiber 216 and on the body of the probe 210. In some cases, the highest temperature can be taken from a collection of marks.

The luminescent marks can include one or more of a crystalline phosphor ceramic, an organic component, an arrangement of one or more quantum dots (e.g., in a binding agent), a nanostructure, among other types and arrangements. The luminescent marks can be on the order of about 100 microns across in diameter each.

Depending on the specific materials of the luminescent marks 220, a variety of types of luminescence can be used to measure and control the temperature in the probe. For example, photoluminescence, is a light emission as a result of absorption of photons that includes: fluorescence with typical lifetime of nano- and microseconds and phosphorescence where light emits from milliseconds to hours.

In addition to photoluminesence, other types of luminescence, such as candoluminescence and thermoluminescence can also be used. Thermoluminescence can occur where a solid, such as a crystal, stores incident light energy, and then glows in the visible spectrum when it is later heated. That is, thermoluminescent materials can be heated and cooled depending on the temperature proximate to the tissue. Thermoluminescent materials can additionally include materials such as storage phosphors or electron trapping materials, where pulses of infrared light can release stored energy in the form of visible light, where the light intensity varies with temperature.

Thermoluminescence also known as thermally stimulated luminescence refers to the process in which a solid, usually in crystalline form, emits light while being heated following excitation. When such a crystal is irradiated, a portion of the absorbed energy can be stored in the lattice and recovered later in the form of visible light emission if the material is heated. The emitted light intensity is commonly composed of one or more glow peaks.

In an example, a storage phosphor or electron trapping material can be used as the luminescent marker(s). Electron trapping or photostimulable phosphors, also called storage phosphors, are compounds that are capable of absorbing and storing energy from visible light or X rays. They can be stimulated subsequently to release energy in the form of visible light. The storage phosphors emission intensity is sensitive to the environment temperature. The light stimulation can be performed over the probe by a visible light source or a near infrared signal.

In additional examples, long afterglow (e.g., persistent) phosphors can be used, such as SAO phosphors, CaS phosphors, SAO25 phosphors, LAO phosphors, CAO phosphors, YOS phosphors, and others. In this case, the afterglow or decay time of luminescent materials can be monitored. For example, short light bursts of a certain wavelength (e.g., about a few milliseconds of blue light) as excitation energy from the laser source or the visible light source. A “certain wavelength” based on the material used as the luminescent mark can then be selected. For example, blue light could be used where it excites photoluminescence of the particular material the luminescent is made of, such as a crystal or quantum dot that responds to blue light wavelength. Based on how the luminescent mark excites, the decay emission can be monitored, and from this the temperature at that location can be determined. In this way, the back-reflected signal can show how the luminescence decays, thereby giving an indication of temperature at the mark. Decay time of luminescence may be practical for checking the temperature. The decay depends on the temperature. The decay might only last a few milliseconds too (where the pulse was a few milliseconds), depending on various factors.

Based on the particular surgical tools being used, the procedure being done, or the anatomical area being treated, the surgeon may desire to monitor a specific temperature range while using the system 200. In this case, luminescent marks that produce luminescence correlated to this temperature range can be used.

In an example, luminescent marks can be used that are continuously excited with the visible light source. In this case, the luminescent color and wavelength intensity can change based on the ambient temperature.

In an example, phosphor thermometry can be employed to measure a temperature over a wide range, such as from 0° C. to up to 1400° C., depending on material properties of a material used for a luminescent mark. A phosphor thermometry technique can be particularly advantageous in an application in which temperature is difficult to measure using conventional techniques.

During operation, light from the light source 214 can be directed at the luminescent mark 220 to produce a specific luminescence. The produced luminescence can be monitored to determine the temperature changes during surgery. Based on the materials for the luminescence marks, the wavelengths of luminescence can be tailored so specific temperature information is gleaned.

The luminescence from the luminescent mark (whether directly entering the laser fiber through the side, through the end, reflected off tissue, or elsewhere) can be configured, based on the luminescent materials, to not interfere with other light from the light source that is used, for example, for surgical view of the tissue. In an example, light from the light source 214 reflected from the targeted tissue during surgery can be used to determine the composition of the kidney stone or tissue, or simply to see the surgical field. Reflections of light from targeted tissue such as a kidney stone could be reflections of the visible light in from the light source, about 380 nm to 700 nm in wavelength.

In this case, the luminescent mark can be made to emit or glow in a non-interfering range, such as at about 800 nanometers (nm). Thus, light from the luminescent mark and any other reflected light from the medical procedure could both use the same conduits to return the light, without interference. The camera 212 can thus pick up and differentiate both types of light. In some examples, time-multiplexing can be leveraged to take turns on which reflected light is analyzed.

In some cases, light from the luminescent marks can overlap in wavelength with other procedure-related light. In this case, the luminescent marks can be configured to have a desired wavelength range. For example, the emitted wavelengths can cover a broad range, such as about 750 nm to about 900 nm. In some cases, a narrow emission from the luminescent marks can be monitored, such that peaks at particular wavelengths can be detected relative to other visible light in the area.

In the context of a medical device, before or at the beginning of a procedure, desired temperatures can be determined, and wavelengths from the luminescent marks can be read to determine the wavelength at certain temperatures. In an example, the temperature correlating to each luminescent mark at a given color or frequency can be determined empirically to develop a table or database. Thus, later in the procedure, received luminescent mark wavelengths or colors can be compared to the empirically determined table or database to determine the temperature at a time stamp during the procedure.

In some cases, a single crystal luminescent mark (or a single luminescent material or component) can be used. In some cases, a mixture of two or more luminescent materials can be used. In this case, the materials can each have a different temperature dependence. In this case, a mixture of or more luminescent materials, each having a different temperature dependence, can be used. Changes in emission color can be detected based on the relative temperature quenching.

For example, a mixture of luminescent materials can be used. The materials can have different temperature quenching of emission where the luminescence intensity of each single composition depends on ambient temperature in different way. The total emission spectrum of the mixture can include combinations of specific spectra of each of the materials presented in the mixture. Due to the difference in temperature quenching of luminescent intensities of each of the materials, the total emission spectra, and color of the mixture, can depend on ambient temperature. The emission spectra variations correlated with ambient temperature can be measured by spectrometer and analyzed by spectral analyzer based on preliminary calibration data for spectra shape versus temperature. This can be a multi-color implementation of the technique based on the intensity ratio of emissions from different distinct spectrum.

An example of the method of temperature detection based on analyses of intensity ratios of two or more separate emission spectra (lines) related to the different activator in one host material; change in temperature is reflected by the change of the phosphorescence spectrum. For example, oxysulfide materials multi-doped by Ln³⁺ (Ln=Nd, Sm, Eu, Dy, Ho, Er, Tm, Yb) that exhibit several different emission lines related to different activators, each having a different temperature dependence.

In another example, two luminescent marks can be used, one for blue light and one for red light. This can be created using a mixture of two different materials, where each material depends on the ambient temperature differently from each other. At a first temperature, such as 20° C., a first combination of intensities would be detectable. At a second temperature, such as 50° C., a second combination of intensities would be detectable. A change in emission color could be detected between the two temperature, based on temperature quenching (e.g., decreased luminescence). Intensity can be dependent on temperature. Such a mixture can include two or more different crystallized materials, each having different emission wavelengths and colors.

Mixing luminescent materials can provide different wavelengths of luminescence. Emission intensity is based on the mixture. In an example, one material can emit in green light, and another can emit in red light. In some cases, the emission is narrow enough to have separated lines. In some cases, the green emission can be strongly is dependent on temperature. For example, if temperature drops 10 degrees, then the green emission might drop some amount, such as about 10%. But the red light may not be as sensitive as the green light, and with the same 10 degree decrease, the red light might only drop 1% or 2%. In this case, the signal would become more dominantly red, since the red light is not decreasing at the rate that the green decreases. In this way, two or more materials being mixed can give indications of the temperature.

In mixtures of luminescent mark materials, leveraging one component that is more sensitive to temperature (e.g., decays faster based on temperature), and one component that is relatively more stable, can be useful. This can allow for a clear change in the color of the light as temperature changes. That color can be compared to previously-detected colors, and color-to-temperature dependence data can be collected. Empirical data can be collected to correlate colors with actual temperatures.

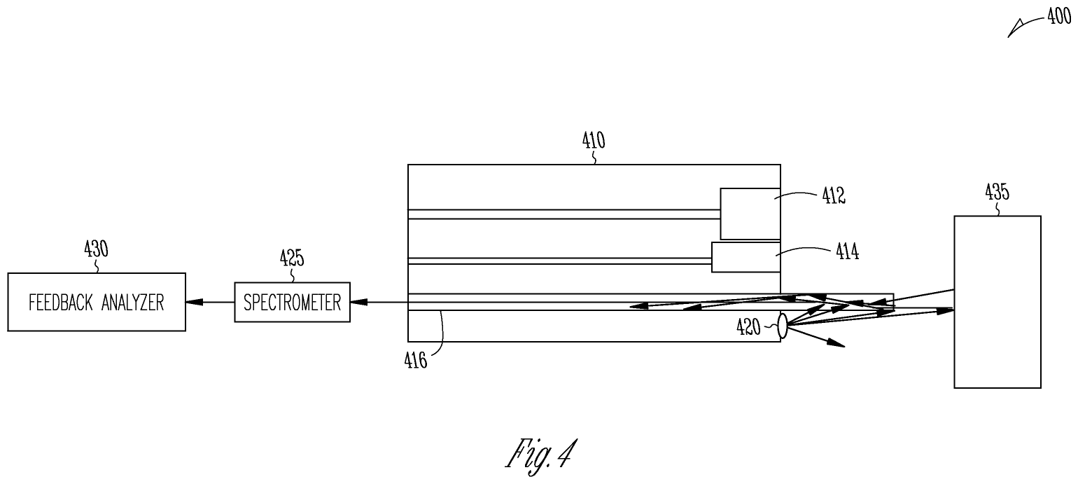

Luminescent marks, such as luminescent mark 220, can be used in conjunction with signal processing equipment, such as spectrometers and feedback analyzers, as discussed with reference to FIGS. 3 and 4 below.

FIG. 3 depicts a surgical system 300, which can include a probe 310, camera 312, light source 314, laser fiber 316, luminescent mark 320, and feedback analyzer 330. The components of system 300 can be similar to, and connected in a similar fashion, to the corresponding components discuss with reference to FIGS. 1A-1B above, except where otherwise noted.

In the system 300, the camera 312, light source 314, and the laser fiber 316 can extend at least partially within the probe 310. The luminescent mark 320 can be situated on the laser fiber 316. When in use, the laser fiber 316 can be energized by a light source 314, and the luminescent mark 320 can be energized by the light source 314. The camera 312 can capture produced luminescence, which can be correlated to a temperature of the system 300 by use of the feedback analyzer 330.

Similar to the system 100 above, the light source 314 can be, for example, a visible light source to illuminate the tissue, and to allow for camera images, in addition to activating the luminescent mark 320. The camera 312 can capture the luminescence and visible images. The produced luminescence can be processed by signal processing circuitry in the feedback analyzer 330 such as to compare to a previously determined luminescence-to-temperature relationship to determine the temperature of the tissue. In some examples, light from the luminescent mark can be captured from the laser fiber itself.

The feedback analyzer 330 can be coupled to one or more components of the system 300 so as to receive signals indicative of luminescent activity from the luminescent mark 320. For example, the feedback analyzer 330 can be coupled to the camera 312. In this case, the camera 312 can capture one or more optical signals of luminescence and convey them to the feedback analyzer. In some cases the feedback analyzer 330 can be coupled to the laser fiber 316, so as to receive indications of luminescence interacting with the laser fiber 316.

The feedback analyzer 330 can include a controller. Such a controller can be a programable controller, such as a single or multi-board computer, a direct digital controller (DDC), a programable logic controller (PLC), or the like. In other examples the controller can be any computing device, such as a handheld computer, for example, a smart phone, a tablet, a laptop, a desktop computer, or any other computing device including a processor, memory, and communication capabilities.

The feedback analyzer 330 can additionally include a user interface. The user interface can be any display and/or input device. For example, user interface can be a monitor, keyboard, and mouse in one example. In other examples, user interface can be a touch screen display. In yet another example, user interface can provide lights, buttons, and/or switches. The controller and user interface can include machine readable medium. The terms “machine readable medium” may include any medium that is capable of storing, encoding, or carrying instructions for execution by the device and that cause the device to perform any one or more of the techniques of the present disclosure, or that is capable of storing, encoding or carrying data structures used by or associated with such instructions. Non-limiting machine readable medium examples may include solid-state memories, and optical and magnetic media. Specific examples of machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

In system 300, the detected luminescence signals can be analyzed and processed by the feedback analyzer 330 to determine temperature, for example, by detecting or monitoring parameters of luminescence itself, or determining a chance of monitored luminescence.

Luminescence detection can be compared with stored information about connecting luminescence and temperature, such as to correlate the luminescence to temperature. The stored information about the relationship between luminescence and temperature can take various forms, such as a lookup table, a fitted function, or other data.

Alternatively, the processor can receive the luminescence data and generate an appropriate curve or chart comparing luminescence to temperature, and compare the generated luminescence-temperature curve to a whole library of luminescence-temperature curves, or to a sub-set of luminescence-temperature curves. In some cases, a desired threshold value of change in temperature can be determined, and compared to the produced luminesce.

In any case, the determination by the processor can then be conveyed to the user. In some cases, the verification of the temperature can be conveyed through a user interface. In other cases, the verification can be conveyed to the user via a small light, such as an LED light, on the console. In other cases, the verification can be conveyed to the user via a sound or tone.

FIG. 4 depicts a surgical system 400, which can include a probe 410, camera 412, light source 414, laser fiber 416, luminescent mark 420, spectrometer 425, and feedback analyzer 430. The components of system 400 can be similar to, and connected in a similar fashion, to the corresponding components discuss with reference to FIGS. 1A-1B above, except where otherwise noted.

In the system 400, the camera 412, light source 414, and the laser fiber 416 can extend at least partially within the probe 410. The luminescent mark 420 can be located on the probe 410 end. This arrangement can allow for detection of light and temperature at a

When in used, the laser fiber 416 can be energized by a light source 414, and the luminescent mark 420 can be energized by the light source 414. The camera 412 can capture produced luminescence, which can be correlated to a temperature of the system 400 by used of the feedback analyzer 430 and spectrometer 425.

Similar to the system 100 above, the light source 414 can be, for example, a visible light source to light up the tissue, and to allow for camera images, in addition to activating the luminescent mark 420. The camera 412 can capture the luminescence and visible images. An optical signal from the produced luminescence can be sent to the spectrometer 425 and processed. This can be further processed by signal processing circuitry in the feedback analyzer 430 such as to compare to a previously determined luminescence-to-temperature relationship to determine the temperature of the tissue. In some examples, light from the luminescent mark can be captured from the laser fiber itself.

The spectrometer 425 can be used to separate and measure spectral components of optical signals produced by luminesce of the luminescent marks 420. The spectrometer can, for example, measure a continuous variable of a luminescence where the incoming spectral components are mixed. The spectrometer 425 can, for example, pick up and detect the wavelengths of luminesce occurring over time. In some cases, this can be used to produce a graph of time versus luminesce. Such data can be passed onto the feedback analyzer 430 to determine changes in temperature.

### Various Notes & Examples

Each of these non-limiting examples can stand on its own, or can be combined in various permutations or combinations with one or more of the other examples.

Example 1 is a medical device comprising: a medical probe, including a distal portion configured for at least partial insertion into a patient; and one or more luminescent marks, located on the distal portion of the probe, and having a luminescent characteristic correlative to temperature, when illuminated, to provide an indication of the temperature at an internal site of the patient.

In Example 2, the subject matter of Example 1 optionally includes a light source for providing light to the one or more luminescent marks.

In Example 3, the subject matter of any one or more of Examples 1-2 optionally include a sensor for detecting a luminescence response signal from the one or more luminescent marks.

In Example 4, the subject matter of Example 3 optionally includes wherein the sensor comprises at least one of a camera or a spectrometer.

In Example 5, the subject matter of any one or more of Examples 3-4 optionally include a signal analyzer coupled to the sensor for analyzing the detected luminescence response and generating a resulting indication of the temperature at the internal site based at least in part on the analysis and the luminescent characteristic associated with the one or more luminescent marks.

In Example 6, the subject matter of any one or more of Examples 1-5 optionally include a laser system having a controller and a laser fiber for treating a target at an internal site of the patient.

In Example 7, the subject matter of Example 6 optionally includes wherein the one or more luminescent marks are located on a distal portion of the laser fiber.

In Example 8, the subject matter of any one or more of Examples 6-7 optionally include wherein the controller is configured to adjust at least one setting of the laser system based at least in part on the indication of the temperature at the internal site.

In Example 9, the subject matter of any one or more of Examples 6-8 optionally include wherein the one or more luminescent marks are located in an optical core, a cladding layer, a buffer layer and/or a jacket layer of the laser fiber.

In Example 10, the subject matter of any one or more of Examples 1-9 optionally include wherein the one or more luminescent marks are located on an end of the medical probe.

In Example 11, the subject matter of any one or more of Examples 1-10 optionally include wherein at least one of the one or more luminescent marks comprises a piece of crystal, a crystalline material, a polycrystalline material, an organic component, an arrangement of quantum dots, or combinations thereof.

In Example 12, the subject matter of any one or more of Examples 1-11 optionally include wherein at least one of the one or more luminescent marks comprises crystalline phosphor ceramics, organic components, quantum dots, nanostructures, or combinations thereof.

In Example 13, the subject matter of any one or more of Examples 3-12 optionally include wherein the detected luminescence response signal comprises photoluminescence, candoluminescence, thermoluminescence, or combinations thereof.

In Example 14, the subject matter of any one or more of Examples 1-13 optionally include nm or less.

In Example 15, the subject matter of any one or more of Examples 1-14 optionally include a feedback analyzer coupled to the sensor, wherein the feedback analyzer includes signal processing circuitry.

In Example 16, the subject matter of Example 15 optionally includes a controller configured for interpretation of luminesence signals.

Example 17 is a method of monitoring a temperature near a target comprising: inducing luminescence of one or more luminescent marks, the one or more luminescent marks having a luminescent characteristic correlative to temperature and being located in proximity to the target; detecting the induced luminescence; and determining the temperature of the target based at least in part on the detected luminescence and the luminescent characteristic associated with the one or more luminescent marks.

In Example 18, the subject matter of Example 17 optionally includes wherein determining the temperature of the target comprises correlating the detected luminescence to temperature based on a lookup table.

In Example 19, the subject matter of any one or more of Examples 17-18 optionally include wherein the detected luminescence comprises photoluminescence, candoluminescence, thermoluminescence, or combinations thereof.

In Example 20, the subject matter of any one or more of Examples 17-19 optionally include adjusting at least one setting associated with a treatment device based at least in part on the determined temperature.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A medical device comprising:
a medical probe, including a distal portion configured for at least partial insertion into a patient; and
one or more luminescent marks, located on the distal portion of the probe, and having a luminescent characteristic correlative to temperature, when illuminated, to provide an indication of the temperature at an internal site of the patient.

2. The device of claim 1, further comprising a light source for providing light to the one or more luminescent marks.

3. The device of claim 1, further comprising a sensor for detecting a luminescence response signal from the one or more luminescent marks.

4. The device of claim 3, wherein the sensor comprises at least one of a camera or a spectrometer.

5. The device of claim 3, further comprising a signal analyzer coupled to the sensor for analyzing the detected luminescence response and generating a resulting indication of the temperature at the internal site based at least in part on the analysis and the luminescent characteristic associated with the one or more luminescent marks.

6. The device of claim 1, further comprising a laser system having a controller and a laser fiber for treating a target at an internal site of the patient.

7. The device of claim 6, wherein the one or more luminescent marks are located on a distal portion of the laser fiber.

8. The device of claim 6, wherein the controller is configured to adjust at least one setting of the laser system based at least in part on the indication of the temperature at the internal site.

9. The device of claim 6, wherein the one or more luminescent marks are located in an optical core, a cladding layer, a buffer layer and/or a jacket layer of the laser fiber.

10. The device of claim 1, wherein the one or more luminescent marks are located on an end of the medical probe.

11. The device of claim 1, wherein at least one of the one or more luminescent marks comprises a piece of crystal, a crystalline material, a polycrystalline material, an organic component, an arrangement of quantum dots, or combinations thereof.

12. The device of claim 1, wherein at least one of the one or more luminescent marks comprises crystalline phosphor ceramics, organic components, quantum dots, nanostructures, or combinations thereof.

13. The device of claim 3, wherein the detected luminescence response signal comprises photoluminescence, candoluminescence, thermoluminescence, or combinations thereof.

14. The device of claim 1, wherein each of the one or more luminescent marks comprises a diameter of about 100 nm or less.

15. The device of claim 1, further comprising a feedback analyzer coupled to the sensor, wherein the feedback analyzer includes signal processing circuitry.

16. The device of claim 15, further comprising a controller configured for interpretation of luminesence signals.

17. A method of monitoring a temperature near a target comprising:
inducing luminescence of one or more luminescent marks, the one or more luminescent marks having a luminescent characteristic correlative to temperature and being located in proximity to the target;
detecting the induced luminescence; and
determining the temperature of the target based at least in part on the detected luminescence and the luminescent characteristic associated with the one or more luminescent marks.

18. The method of claim 17, wherein determining the temperature of the target comprises correlating the detected luminescence to temperature based on a lookup table.

19. The method of claim 17, wherein the detected luminescence comprises photoluminescence, candoluminescence, thermoluminescence, or combinations thereof.

20. The method of claim 17, further comprising adjusting at least one setting associated with a treatment device based at least in part on the determined temperature.
